(19) United States
(12) Patent Application Publication
(10) Pub. No.: US 2002/0010132 A1
(43) Pub. Date: Jan. 24, 2002

(54) COMPOSITIONS OF MURAMYL PEPTIDES INHIBITING THE REPLICATION OF HIV

(75) Inventor: Georges BAHR, Puteaux (FR)

Correspondence Address: Birch, Stewart, Kolasch & Birch, LLP, ., 8110 Gatehouse Road, Suite 500 East, Falls Church, VA, 22040, US, mailroom@bskb.com, 703-205-8000, 703-205-8050

(73) Assignee: VACSYN S.A., Paris (FR)

(21) Appl. No.: 08/809,650

(22) Filed: Jun. 13, 1997

(30) Foreign Application Priority Data
Sep. 26, 1995 (FR) PCT/FR95/01239
Sep. 26, 1994 (FR) 94/11460

(51) Int. Cl. A61K 38/00

(52) U.S. Cl. 514/12

(57) ABSTRACT

### Abstract of Disclosure

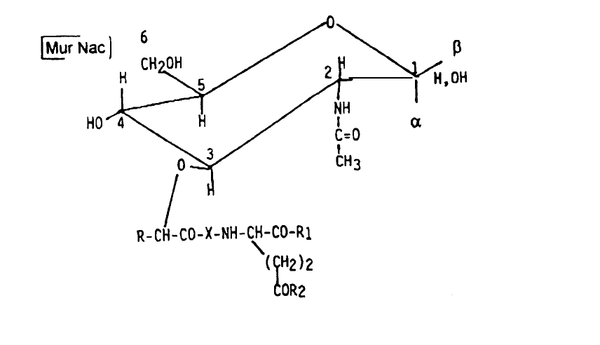

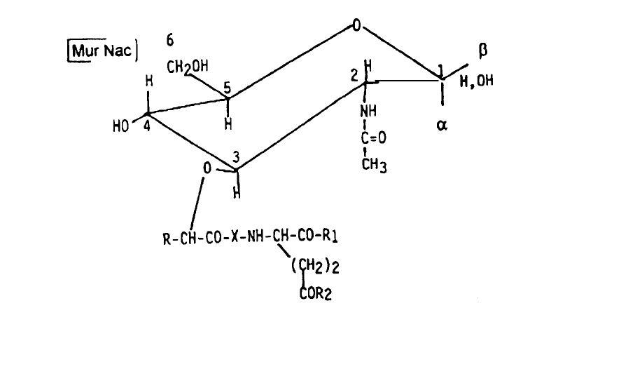

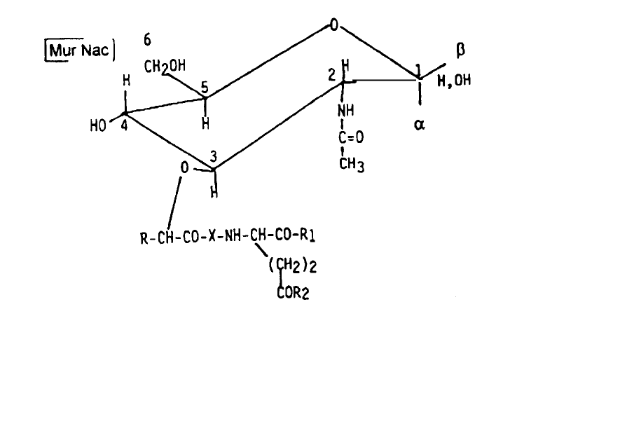

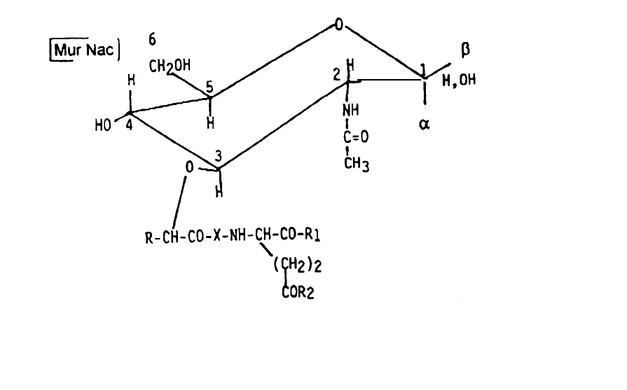

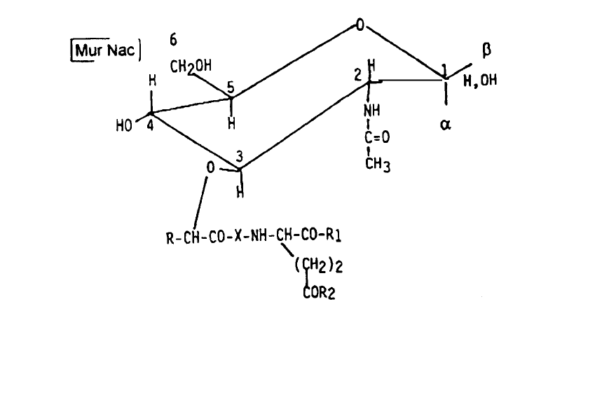

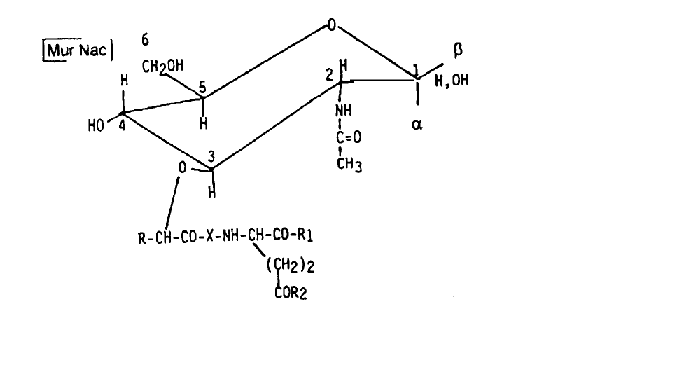

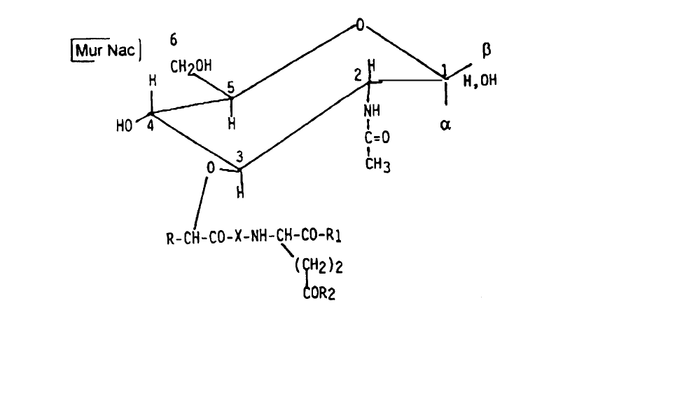

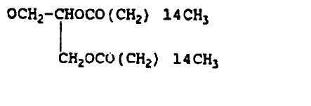

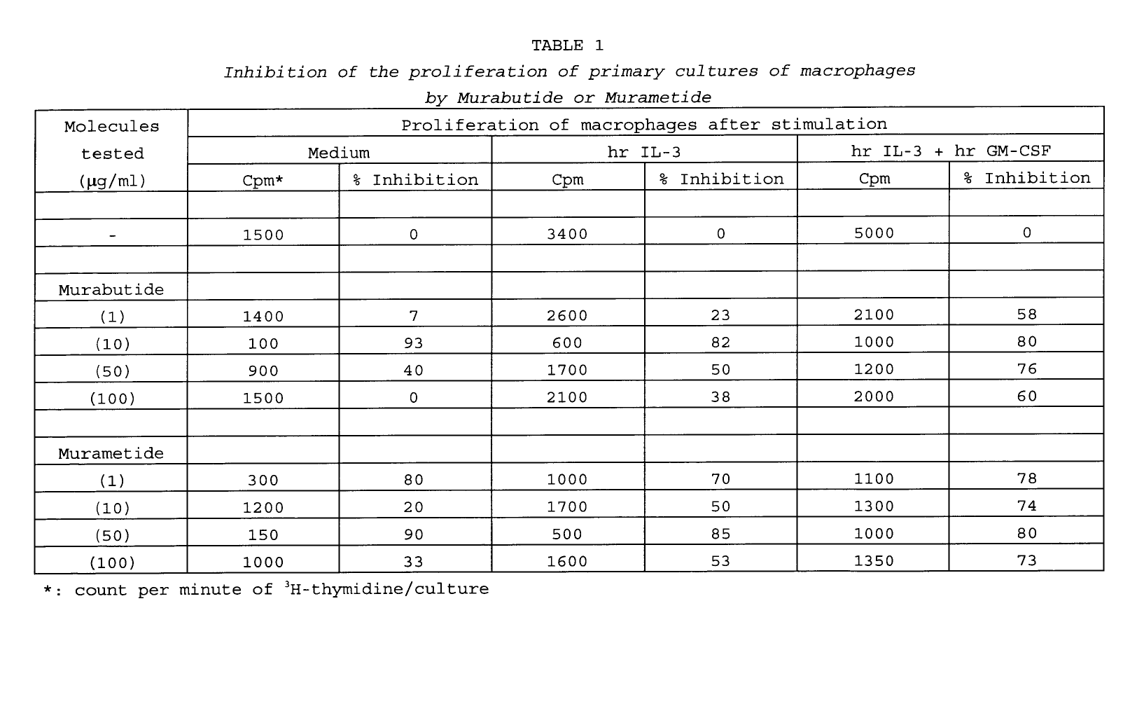

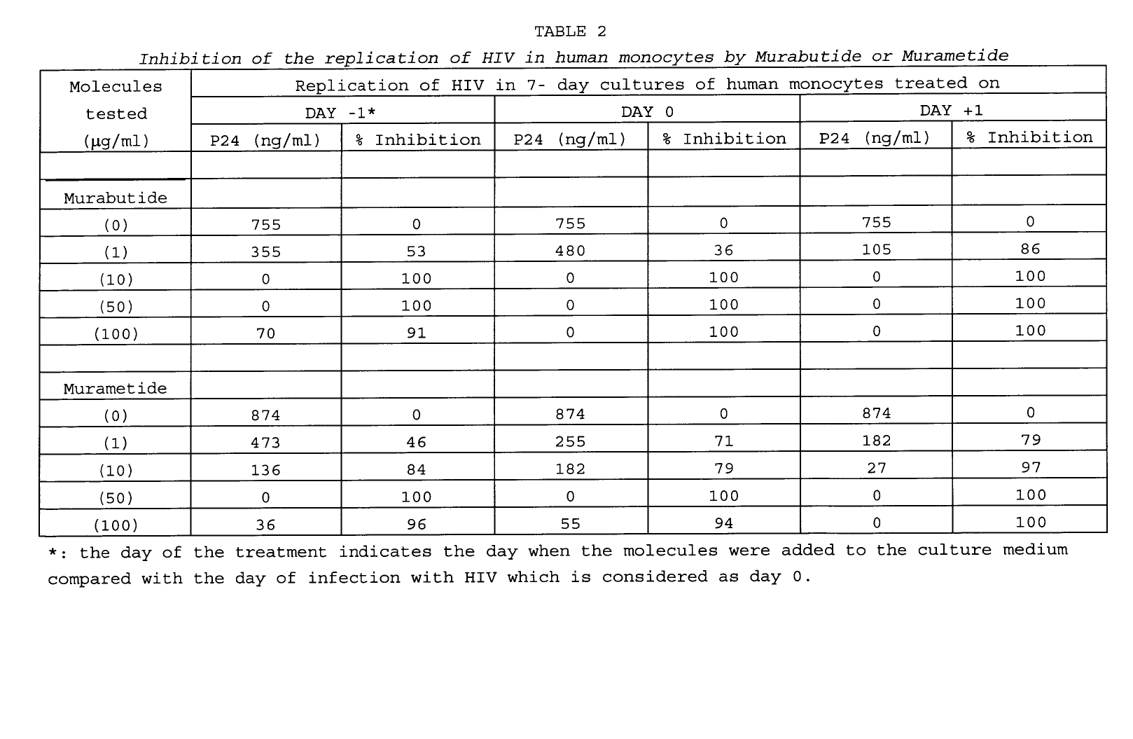

The use of non-toxic muramyl peptides, particularly Murabutide and Murametide, to prepare drugs for inhibiting HIV replication in humans is disclosed. Said muramyl peptides are capable of up to 100% inhibition of retroviral replication in primary host monocyte cultures.

### Detailed Description

Acquired immunodeficiency syndrome (AIDS) is a devastating disease caused by infection by the HIV retrovirus. A lot of effort has been devoted to finding medicaments capable of inhibiting the replication of the virus. However, few significant successes have been obtained so far. Although HIV can infect many different cells, the disease is predominantly caused by the destruction and/or the dysfunction of a subpopulation of lymphocytes called helper T cells. The persistence of the infection by the virus has not long ago been attributed to its capacity to infect another major cell population, the monocyte/macrophage line, which is thought to serve as a reservoir for a continuous release of the virus. The major role played by this HIV line in the persistence and the progression of the disease has been explained by 1) the isolation of monocytotropic variants of HIV from the circulating blood leukocytes and tissue macrophages of infected subjects at all stages of the infection (J. Virology, ; Vol. 65, pages 356-363, 1991) and, 2) the direct correlation between an absence of systemic immunity dysfunction in the infected host and an absence of viral replication in the monocyte/macrophage line (J. infectious diseases, Vol. 168, pages 1140-1147, 1993). Furthermore, the inhibition of a virus-producing infection in the monocytes appears to be linked to a large extent to the inhibition of the monocytic proliferation, which suggests that the replication of the virus depends on a preliminary obligatory stage of high proliferation of the monocytic cell. Thus, the proliferation of this population is thought to be an obligatory passage for the manifestation of the infectious HIV character. Thus, the hypothesis has been formulated that substances capable of inhibiting monocytic replication might also inhibit the replication of HIV (J. Clinical Investigation, Vol. 89, pages 1154-1160, 1992).

Muramyl peptides are synthetic copies of the bacterial wall and have been found to be capable of highly numerous immunopharmacological activities on the monocyte/macrophage line (Federation proceedings, Vol. 45, pages 2541-2544, 1986). Furthermore, the initial molecule N-acetyl-muramyl-L-alanyl-D-Isoglutamine (Nac-Mur-L-Ala-DisoGln) also called Muramyl dipeptide or MDP, has been described to be capable of inhibiting the proliferation of guinea pig macrophages (Cellular Immunology, Vol. 89, pages 427-438, 1984). In another study using established lymphocyte cell lines or established lines of monocyte-type cells, MDP was found to be endowed with the capacity of partially inhibiting the replication of HIV when it is used in vitro at very high doses of 1000 μg/ml (AIDS Research and Human Retroviruses, Vol 6, pages 393/394, 1990). However, besides the fact that the use of MDP in human clinical medicine is difficult to envisage because of the side effects which it induces, the observed effects, even at these high doses in the experimental system used, would not presage any therapeutic efficacy towards HIV infection. Lazdins et al (AIDS Research and Human Retroviruses, Vol. 6, pages 1157-1161, 1990) have shown, in vitro, similar properties of inhibition of the replication of HIV for a muramyl peptide having a better therapeutic index than MDP : MTP-PE. This molecule, in free form, was added repeatedly, before and after HIV infection, to cultures of macrophages derived from cultured human monocytes. However, it was able to induce, under these conditions, only a partial reduction in viral replication. It should be emphasized that MTP-PE was not capable, either in the free form or incorporated into liposomes, of causing total suppression of viral replication. In addition, its activity can be exerted only if this component is present on the day the cell culture is infected by the virus. If the compound is added a day before or 4 days after the culture, its activity is minimal.

These results only make more surprising those which have been obtained with another category of muramyl peptides, which have been found to allow complete inhibition of the proliferation of HIV, especially in primary cultures of moncytes, and this at much lower doses. Their lower toxicity coming on top of these favorable effects, therefore make them suitable for the preparation of medicaments capable of preventing or treating AIDS and/or of the related syndromes.

The invention relates more particularly to the use, for the preparation of medicaments inhibiting the replication of acquired immunodeficiency retroviruses in man or those of mammals which they are capable of infecting, of a muramyl peptide of formula:

in which the group R is a hydrogen or a methyl group; X is an L-alanyl, L-threonyl or L-lysyl residue, and R1 is a hydroxyl, an amino or an O(CH2)ₓH group with x=1,2,3 or 4, R2 is, independently of R1, a hydroxyl, an amino or an O(CH₂)ₓH group with x=1,2,3 or 4, or a group

it being understood that, when X is an L-alanyl residue, at least one of these two groups R1 and R2 is still an O(CH2)ₓH group as defined above, and that R2 cannot be:a group

A subcategory of muramyl peptides preferred for the production of the abovementioned medicaments consists of hydrophilic muramyl peptides corresponding to the abovementioned general formula in which the R group is a hydrogen or a methyl group; X is an L-alanyl or L-threonyl residue, and R1 and R2 are, independently of each other, hydroxyl, amino or O(CH2)ₓH groups with x=1,2,3 or 4, it being understood that, when X is an Lresidue, at least one of these two groups R1 and R2 is still an O(CH2)ₓH group as defined above.

Preferred compounds for use according to the invention are Murabutide (Nac-Mur-L-Ala-DGln OₙC₄H₉) and Murametide (Nac-Mur-L-Ala-DGln OMe). These molecules exhibit an excellent activity profile in man; they are free of side effects and have demonstrated their very good tolerance, during clinical trials carried out in healthy volunteers and in cancer subjects.

Another preferred subcategory is that corresponding to the abovementioned general formula and in which R2 is a group

for example one of the following two compounds:

- Nac-Mur-L-Lys D-iso-Gln-glycerol, sn dipalmitoyl, and

- Nac-Mur-L-Thr D-isoGln-glycerol sn dipalmitoyl.

It is in this regard remarkable that the abovementioned muramyl peptides are capable, at relatively low concentrations, of exerting a complete inhibition, up to 100%, of the proliferation of HIV, in primary cultures of monocytes, and this more particularly in the experimental procedures which will be referred to hereinafter.

It is particularly important to note that the manifestation of the inhibitory effect of these muramyl peptides towards retroviral replication is not linked to a simultaneity of infection of the monocytes and of treatment of the latter with these muramyl peptides.

Additional characteristics of the invention will appear further in the description which follows, of the biological effects exerted by two preferred muramyl peptides towards the replication of HIV in primary cultures of human monocytes collected from healthy volunteers.

In example 1, Murabutide and Murametide demonstrated their capacity to inhibit the proliferation of macrophages in culture. For that, monocytes collected from a donor are cultured for 5 days either a) without stimulation (so as to evaluate their spontaneous proliferation level) or b) in the presence of human recombinant interleukin-3 (hr IL-3) or c) in the presence of both hr IL-3 and hr GM-CSF human recombinant granulocyte-macrophage colony stimulating factor. These two treatments make it possible to obtain a high level of proliferation. The compounds of the invention are added to the culture medium a day before the addition of tritiated thymidine (³H-thymidine). The dividing cells incorporate this thymidine. The cells (which have differentiated into macrophages during the duration of the culture) are recovered and washed, and the proliferation level is evaluated by measuring, in a beta counter, the quantity of ³H incorporated according to conventional methods as described in Blood, Vol. 76, pages 1490-1493, 1990. The results are presented in Table 1 and show that the two derivatives are capable, even at the dose of 1 μg/ml, of inhibiting the proliferation of macrophages stimulated with IL-₃ or the combination IL-₃/GM-CSF. The effect of inhibition of spontaneous proliferation was observed with 10 μg/ml of Murabutide and 10 or 50 μg/ml of Murametide.

Example 2 demonstrates the effect of Murabutide and Murametide on the level of replication of HIV in primary cultures of human monocytes collected from healthy volunteers. Monocyte cultures were infected on day 0 with an HIV source (HTLV III Ba-L) which exhibits a tropism for the monocytes. Some cultures were treated with different concentrations of the compounds either 1 day before, or the same day, or 1 day after inoculation with HIV. The replication of the virus was evaluated on day 7 by measurement of the quantity of viral protein P24 in the supernatants as described in Blood, Vol. 76, page 1490-1493, 1990. The results presented in Table 2 show clearly that the treatment with Murabutide at a concentration of 10 to 50 μg/ml completely inhibits viral replication whether the treatment has been performed on day -1, on day 0 or on day ‘in relation to the infection. Similarly, the treatment with Murametide made it possible to observe a highly significant suppression of viral replication and this effect is 100% at the dose of 50 μg/ml regardless, here also, of the amount of the treatment.

These results are the first described which have made it possible to obtain a complete inhibition, by a muramyl peptide, of the replication of HIV in human monocytes. It should be emphasized that the inhibition is obtained when the compound is added to the culture only once and even after infection by HIV.

The preceding data show that the muramyl peptides of the invention can be applied to the preparation of medicaments applicable to the prevention or treatment of AIDS, or related syndromes, for example Kaposi's sarcoma.

The invention is also applicable to the preparation of medicaments in which the muramyl peptides are used in combination with other therapeutic agents used to prevent or inhibit the proliferation and the diffusion of HIV in man. Among these agents, there may be mentioned the α-, β- and Υ-interferons and GM-CSF.

The molecules of the invention may be used in human clinical medicine either for preventive purposes in at-risk subjects, or for curative purposes in seropositive individuals before the appearance of clinical signs or in patients having developed manifestations of AIDS. The therapeutic doses of the muramyl peptide (for example Murabutide or Murametide) to be administered either alone, or in combination with antiviral treatments, particularly cytokines, are between 1 μg and 500 μg/kg/day. The administrations may be given by the systemic route, by subcutaneous or intravenous injection or by infusion. The treatment may consist of daily administrations or administrations at a few days" interval and may be extended by a week to several months depending on the observed effect.

In the case of seropositive or sick individuals, the treatment should be prolonged until there is no detection of antigen or of viral genes in the serum or the cells of the infected individual, respectively. In the case of at-risk individuals, the preventive treatment should be applied during the period where a risk of infection exists.

The molecules of the invention as well as the other molecules of the family of muramyl peptides may also be used as laboratory reagents so as to allow the evaluation, as anti-HIV agents, of drugs presumed to have antiviral activity. Thus suboptimal doses of muramyl peptides could be used in combination with another agent to detect a potential activity of the latter.

This type of reagent could be used in experimentation systems in vitro using monocyte/macrophage cultures as described in this patent or methods of evaluation in vivo including the use of SCID mice.

Claims

25. A process for inhibiting the replication of acquired immunodeficiency retroviruses in man or in those animals which said retroviruses are capable of infecting, which comprises administering as a principal ingredient to said man or said animals in need of such treatment an effective amount of a muramyl peptide of formula:
in which the group R is a methyl group; X is an L-alanyl residue, and R1 is an O(CH₂)ₓH group with x= 1, 2, 3 or 4, R2 is, independently of R1, either an amino or an O(CH₂)ₓH group with x= 1, 2, 3 or 4, and wherein said effective amount is also an amount that is capable of causing a 100% inhibition of the replication of said retroviruses in primary cultures of monocytes of the host.

26.The process of claim 25, wherein both R1 and R2 are O (CH₂) ₓH groups.

27.The process of claim 25, wherein the muramyl peptide is Murametide.

28.The process of claim 25, wherein the muramyl peptide is Murabutide.

29. The process of claim 25, which is for the prevention or treatment of AIDS or related syndromes.

30.The process of claim 25, which comprises administering said muramyl peptide together with another molecule capable of enhancing the anti-retroviral action of said muramyl peptide.

31. The process of claim 30, wherein the other molecule is a cytokine.

32. The process of claim 30, wherein the other molecule is GM-CSF.

33. The process of claim 30, wherein the other molecule is a protease inhibitor.

34. The process of claim 25, wherein the muramyl peptide has the formula:

35. The process of claim 31, wherein the cytokine is selected from the group of an α-,β- or Υ- interferon.

36. The process of claim 25, which is for the prevention or treatment of Kaposi's sarcoma.

37. A process for inhibiting the replication of acquired immunodeficiency retroviruses in man or in those animals which said retroviruses are capable of infecting, which comprises administering as a principal ingredient to said man or said animals in need of such treatment between 1 to 500 μg/kg/day of a muramyl peptide of formula:
in which the group R is a methyl group; X is an L-alanyl residue, and R1 is an O(CH₂) ₓH group with x=1, 2, 3 or 4, R2 is, independently of R1, either an amino or an O(CH₂) ₓH group with x= 1,2, 3 or 4.

38. A process for inhibiting the replication of acquired immunodeficiency retroviruses in man or in those animals which said retroviruses are capable of infecting, which comprises administering as a principal ingredient to said man or said animals in need of such treatment an effective amount of a muramyl peptide of formula:
in which the group R is a methyl group; X is an L-alanyl residue, and R1 is an O(CH₂) ₓH group with x= 1, 2, 3 or 4, R2 is, independently of R1, either an amino or an O(CH₂) ₓH group with x= 1, 2, 3 or 4, wherein said effective amount is also an amount that is capable of causing a 100% inhibition of the replication of said retroviruses in primary cultures of monocytes of the host, and wherein said inhibitory activity of replication of said muramyl peptide is independent of adjuvant activity.

39. A process for inhibiting the replication of acquired immunodeficiency retroviruses in man or in those animals which said retroviruses are capable of infecting, which comprises administering as a principal ingredient to said man or said animals in need of such treatment between 1 to 500 μg/kg/day of a muramyl peptide of formula:
in which the group R is a methyl group; X is an L-alanyl residue, and R1 is an O(CH₂) ₓH group with x=1, 2, 3 or 4, R2 is, independently of R1, either an amino or an O(CH₂) ₓH group with x= 1,2, 3 or 4 and wherein said inhibitory activity of replication of said muramyl peptide is independent of adjuvant activity.

40. A process for inhibiting the replication of acquired immunodeficiency retroviruses in HIV-infected man or in animals, which comprises administering to said man or said animals in need of said treatment an amount of a principal ingredient which causes 100% inhibition of the replication of said retroviruses in primary cultures of monocytes of the host, wherein said principal ingredient consists of a muramyl peptide of formula:
in which the group R is a methyl group; X is an L-alanyl residue, and R1 is an O(CH₂) ₓH group with x= 1, 2, 3 or 4, R2 is, independently of R1, either an amino or an O(CH₂) ₓH group with x= 1, 2, 3 or 4.
